(19) United States
(12) Patent Application Publication
(10) Pub. No.: US 2005/0192479 A1
(43) Pub. Date: Sep. 1, 2005

(54) Rigid endoscope with fiber optics

(75) Inventors: Thomas Forster, Molln (DE); Gregor Kiedrowski, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/056,573

(22) Filed: Feb. 11, 2005

(30) Foreign Application Priority Data
Feb. 26, 2004 (DE) 10 2004 009 219.2

(51) Int. Cl. A61B 1/00

(52) U.S. Cl. 600/182; 600/130

(57) ABSTRACT

A rigid endoscope (1) including a fiber optics (12) running within a guide tube (5) that is rigidly connected to the endoscope (1), an objective (14) mounted in the guide tube in front of a distal end of the fiber optics, and an ocular (22) mounted in front of the fiber optics' proximal end. The fiber optics (12) rests in an axially displaceable manner in the guide tube (5) and is detachably affixed by its proximal end (15) to the endoscope (8).

The present invention relates to an endoscope defined in the preamble of claim 1.

The image guides in rigid endoscopes typically are relay lens optics, or they are video cameras. In special applications, foremost as regards very thin and long endoscopes, however fiber optics will be advantageous. This is especially the case for ureterorenoscopes which can be advanced through the bladder and the renal duct as far as the kidney. Their stem diameter is at most 3 mm and their length is about 450 mm. When inserted into the human body, they will be bent much and therefore are less suited to incorporating lenses; moreover, because of their small diameter, they cannot receive a video chip. Conventionally, therefore, such endoscopes are fitted with fiber optics.

Both the glass fiber optics and the metallic long stem of the endoscope are rigid. However the thermal expansion coefficients of glass and metal being different, resulting into unequal lengths upon heating, such expansion differentials must be compensated. Furthermore lengthwise shifts take place when bending the endoscope stem where, as is the case conventionally, the fiber optics is cross-sectionally eccentric to the stem.

Consequently known endoscopes offer length compensation between the stem and the fiber optics. The WO 96/05764 document affixes the fiber optic's distal end while holding its proximal end in longitudinally displaceable manner. The DE 19822167 A1 document affixes the proximal end of the fiber optics while allowing axially displacing the distal end zone.

However all known designs share the feature that one or the other end of the optic fiber must be permanently affixed inside the endoscope. Removing the optic fiber for repairs is not an option, and problems are incurred as a result.

The end faces of the fiber optics, as well as the objective's optical surfaces, and the window which is typically configured distally before the objective, may fog over when water vapor or steam penetrates the endoscope and makes it useless. To preclude this eventuality, it is known to hermetically seal the space receiving these optical elements from the ambience, for instance by hermetically sealing the guide tube at both ends with windows. If the seal were defective, and vapor were to enter, no repairs will be possible in the known designs. Then the entire endoscope must be replaced.

The objective of the present invention is to create an endoscope of the above species allowing accessing the optical elements for purposes of repairs.

This problem is solved by the features of claim 1.

The present invention allows removing the fiber optics from the guide tube after said fiber optics has been freed at its proximal end. Thereupon both the fiber optics' end faces and also the optical sub-assembly may be cleaned and be reassembled for re-use. As a result the operating costs of an endoscope of the above species are substantially reduced.

As already mentioned above in relation to the state of the art, length compensation between the endoscope and the fiber optics should be available. Moreover accurate longitudinal positioning of the fiber optics is advantageous on optical grounds. The features of claim 2 are advantageous in this respect. In this manner the fiber optics is axially affixed at its distal end while being resilient at its proximal end, that is, allowing length compensation, while being held against the stop. Therefore good compensation of length is made possible and the fiber optic's distal end is kept in its accurate position.

The objective may be affixed separately from the fiber optics in the endoscope, for instance in the guide tube. However the features of claim 3 are advantageous. As a result the objective together with the fiber optics is made retractable, and hence it may be cleaned very conveniently. The stop assures the accurate position of the objective in front of the window conventionally configured at the distal endoscope end, for instance in the guide tube. In this manner the window is preserved from making damaging contacts and also a constant angle of view is attained.

According to the advantageous features of claim 4, the fiber optics is affixed at its proximal end in a support together with which it may be removed from the endoscope; this feature facilitates handling and safe support of the fiber optics.

Advantageously said support may be designed in the manner of claim 5, namely assuring accurate configuration relative to the ocular and advantageous manufacture of the elastic grip.

Lastly the features of claim 6 are advantageous. The window protects the susceptible proximal fiber optics' end face against making contacts during assembly and against dust.

The drawing shows an endoscope of the present invention in illustrative and schematic manner.

FIG. 1 is an enlarged axial section of the distal endoscope's end zone, and

FIG. 2 is an axial section of the proximal endoscope's end zone.

FIGS. 1 and 2 show an axial section of the distal and proximal end zones of the endoscope 1 of the present invention. Said endoscope comprises an elongated metal stem 2 traversed by a continuous tube 3 constituting an operational duct issuing proximally at an intake 4 serving as an implement passageway. A guide tube 5 distally ending at the stem 2 runs parallel to said operational duct.

The guide tube 5 is enlarged at its distal end on the other side of an offset constituting a stop 6 and is sealed by a window 7.

At its proximal end, the guide tube 5 is deflected laterally and runs in a stub 8 which is oblique to the housing of the endoscope 1 and is affixed, illustratively by fusion, in a borehole 9 of said stub 8.

As shown by FIG. 1, optic fibers 10 are located in the residual cross-section of the stem 2 between the guide tube 5 and the continuous tube 3. FIG. 2 shows that, in the endoscope's terminal zone, the optical fibers 10 run toward a sideways facing hookup stub 11 which can be connected to an omitted light-guide cable for connection to a light source.

As shown in FIG. 1, a fiber optics 12 of glass or quartz fibers is mounted with play in the guide tube 5 and is affixed, for instance adhesively, at its distal end to an objective tube 13. In FIG. 1, the objective 14 is shown illustratively as consisting of two lens elements mounted in the objective tube 13. This objective tube 13 is longitudinally displaceable in the guide tube 5 and it rests by its end face against the stop 6.

As shown in FIG. 2, the fiber optics 12 freely projects beyond the end of the guide tube 5 and is affixed for instance adhesively in a support 15. The support 15 is fitted at its proximal end with a window 16 protecting the distal end face of the fiber optics 12.

In the shown embodiment mode, the support 15 comprises an outer, cylindrical surface and rests in axially displaceable manner in a borehole 17 of the stub 8. A bush 18 is screwed onto the diametrically reduced proximal end piece of the stub 8 and is fitted at its proximal terminal rim with spring clips 19 pointing substantially axially and illustratively being leaf springs. The spring clips 19 are affixed to the bush 18 and resiliently rest against the proximal end face of the support 15. As a result, once the bush 18 has been screwed in place, the support 15 and the fiber optics 12 affixed to this support will be pressed into the distal direction, said fiber optics thereby being held at the distal side, together with the objective tube 13, in its final position against the stop 6.

FIG. 2 shows a much simplified embodiment mode of an ocular housing 21 that receives an ocular lens 22 and that is screwed on the outer surface of the stub 8 fitted with a thread 20. FIG. 1 shows the guide tube 5 hermetically sealed at the distal end by the window 7. Sealing is implemented at the proximal end by screwing on the ocular housing 21 sealed by the lens 22. Additional but omitted O-rings and the like may be used also.

If water vapor/steam were to penetrate the inside space so sealed of the guide tube 5, then when said vapor if cooled would precipitate on the optical surfaces, that is in particular on the end faces of the fiber optics 12, on the inside of the distal window 7, on the surfaces of the optics sub-assembly 14 or also on the proximal window 16. As result the optical system would be blinded.

In the shown design of the present invention, disassembly for purposes of cleaning an repairs is feasible.

First the ocular housing 21 is screwed off. Next the bush 18 together with the spring clips 19 is screwed off. Thereupon the support 15 may be pulled out of the borehole 17. In this process the fiber optics 12, together with the objective tube 13, is pulled out of the guide tube 5. Thereupon all optical surfaces may be properly cleaned. Leakage sources may be looked for and remedied. Moreover other defects, such as lens element shifts, can be corrected. In case of fiber rupture in the fiber optics 12, this fiber optics may be replaced. The subsequent re-assembly is carried out in reverse order.

### BACKGROUND OF THE INVENTION

The image guides in rigid endoscopes typically are relay lens optics, or they are video cameras. In special applications, foremost regarding very thin and long endoscopes, however, fiber optics are advantageously used. This is especially the case for ureterorenoscopes which can be advanced through the bladder and the renal duct as far as the kidney. Their stem diameter is at most 3 mm and their length is about 450 mm. When inserted into the human body, they will be bent substantially and, therefore, are less suited to incorporating lenses; moreover, because of their small diameter, they cannot receive a video chip. Conventionally, therefore, such endoscopes are fitted with fiber optics.

Both the glass fiber optics and the metallic long stem of the endoscope are rigid. However, the thermal expansion coefficients of glass and metal are different, resulting in unequal lengths upon heating, therefore, such expansion differentials must be compensated. Furthermore, lengthwise shifts take place when bending the endoscope stem where, as is the case conventionally, the fiber optics is cross-sectionally eccentric to the stem.

Consequently, known endoscopes offer length compensation between the stem and the fiber optics. International application WO 96/05764 discloses a device that affixes the fiber optic's distal end while holding its proximal end in a longitudinally displaceable manner. German Application DE 19822167 A1 discloses a device that affixes the proximal end of the fiber optics while allowing axial displacement of the distal end zone.

However, all known designs share the feature that one or the other end of the optic fiber must be permanently affixed inside the endoscope. Removing the optic fiber for repairs is not an option, and problems are incurred as a result.

The end faces of the fiber optics, as well as the objective's optical surfaces, and the window which is typically configured distally before the objective, may fog over when water vapor or steam penetrates the endoscope and makes it useless. To preclude this condition, it is known to hermetically seal the space receiving these optical elements from the atmosphere, for instance by hermetically sealing the guide tube at both ends with windows. If the seal were defective, and vapor were to enter, no repairs are be possible in the known designs. The entire endoscope must be replaced.

The objective of the present invention is to create an endoscope of the type discussed above, allowing access to the optical elements for purposes of repairs.

### BRIEF SUMMARY OF THE INVENTION

The present invention allows removal of the fiber optics from the guide tube after the fiber optics have been freed at its proximal end. Thereupon, both the fiber optics' end faces and also the optical sub-assembly may be cleaned and reassembled for re-use. As a result, the operating costs of an endoscope of the type described above are substantially reduced.

As already mentioned above in relation to the state of the art, length compensation between the endoscope and the fiber optics should be available. Moreover, accurate longitudinal positioning of the fiber optics is advantageous on optical grounds. In this manner, the fiber optics is axially affixed at its distal end while being resilient at its proximal end, that is, allowing length compensation, while being held against the stop. Therefore, good compensation of length is made possible and the fiber optic's distal end is kept in its accurate position.

The objective may be affixed separately from the fiber optics in the endoscope, for instance in the guide tube. As a result, the objective together with the fiber optics is made retractable, and hence it may be cleaned very conveniently. The stop assures the accurate position of the objective in front of the window conventionally configured at the distal endoscope end, for instance in the guide tube. In this manner, the window is prevented from making damaging contacts and also a constant angle of view is attained.

The fiber optics is affixed at its proximal end in a support, which together may be removed from the endoscope; this feature facilitates handling and safe support of the fiber optics.

Advantageously, the support may be designed in a manner assuring accurate configuration relative to the ocular and advantageous manufacture of the elastic grip.

The window protects the susceptible proximal fiber optics' end face against making contacts during assembly and against dust.

### BRIEF DESCRIPTION OF THE SEVERAL VIEWS OF THE DRAWINGS

FIG. 1 is an enlarged axial section of the endoscope's distal end zone, and

FIG. 2 is an axial section of the endoscope's proximal end zone.

### DETAILED DESCRIPTION OF THE INVENTION

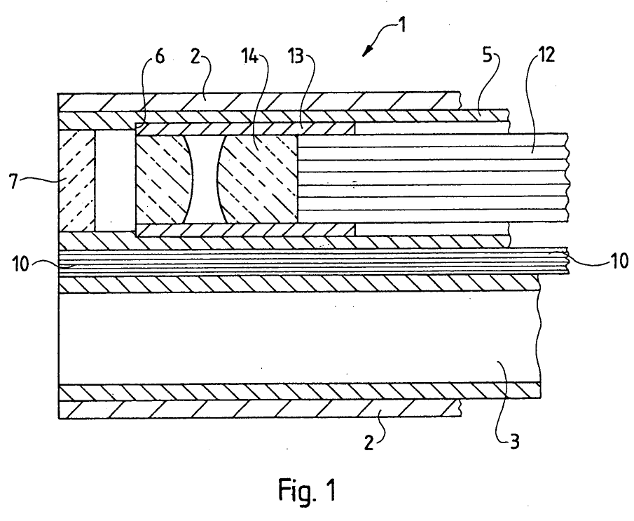

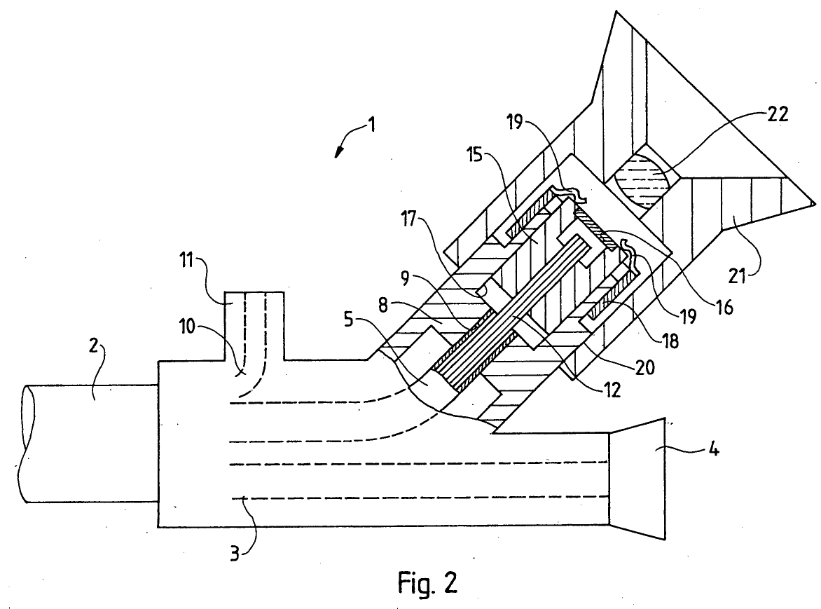

FIGS. 1 and 2 show an axial section of the distal and proximal end zones of the endoscope 1 of the present invention. The endoscope comprises an elongated metal stem 2 traversed by a continuous tube 3 constituting an operational duct issuing proximally at an intake 4 serving as an implement passageway. A guide tube 5 distally ending at the stem 2 runs parallel to said operational duct.

The guide tube 5 is enlarged at its distal end on the other side of an offset constituting a stop 6 and is sealed by a window 7.

At its proximal end, the guide tube 5 is deflected laterally and runs in a stub 8 which is oblique to the housing of the endoscope 1 and is affixed in a borehole 9 of said stub 8.

As shown by FIG. 1, optic fibers 10 are located in the residual cross-section of the stem 2 between the guide tube 5 and the continuous tube 3. FIG. 2 shows that, in the endoscope's terminal zone, the optical fibers 10 run toward a sideways facing hookup stub 11 which can be connected to an omitted light-guide cable for connection to a light source.

As shown in FIG. 1, fiber optics 12 of glass or quartz fibers is mounted with play in the guide tube 5 and is affixed, for instance adhesively, at its distal end to an objective tube 13. In FIG. 1, the objective 14 is shown illustratively as consisting of two lens elements mounted in the objective tube 13. This objective tube 13 is longitudinally displaceable in the guide tube 5 and it rests by its end face against the stop 6.

As shown in FIG. 2, the fiber optics 12 freely projects beyond the end of the guide tube 5 and is affixed for instance adhesively in a support 15. The support 15 is fitted at its proximal end with a window 16 protecting the distal end face of the fiber optics 12.

In the shown embodiment mode, the support 15 comprises an outer, cylindrical surface and rests in axially displaceable manner in a borehole 17 of the stub 8. A bush 18 is screwed onto the diametrically reduced proximal end piece of the stub 8 and is fitted at its proximal terminal rim with spring clips 19 pointing substantially axially and illustratively shown as leaf springs. The spring clips 19 are affixed to the bush 18 and resiliently rest against the proximal end face of the support 15. As a result, once the bush 18 has been screwed in place, the support 15 and the fiber optics 12 affixed to this support will be pressed in the distal direction, the fiber optics thereby being held at the distal side, together with the objective tube 13, in its final position against the stop 6.

FIG. 2 shows a simplified embodiment of an ocular housing 21 that receives an ocular lens 22 and that is screwed on the outer surface of the stub 8 fitted with a thread 20.

FIG. 1 shows the guide tube 5 hermetically sealed at the distal end by the window 7. Sealing is implemented at the proximal end by screwing on the ocular housing 21 sealed by the lens 22. Additional O-rings (not shown) and the like may be used also.

If water vapor/steam were to penetrate the sealed inside space of the guide tube 5, when the vapor cooled, it would precipitate on the optical surfaces, in particular on the end faces of the fiber optics 12, on the inside of the distal window 7, on the surfaces of the optics sub-assembly 14 or also on the proximal window 16. As a result, the optical system would be blinded.

In the illustrated design of the present invention, disassembly for purposes of cleaning and repairs is feasible.

First, the ocular housing 21 is screwed off. Next, the bush 18 together with the spring clips 19 is screwed off. Thereupon the support 15 may be pulled out of the borehole 17. In this process, the fiber optics 12, together with the objective tube 13, is pulled out of the guide tube 5. Thereupon all optical surfaces may be properly cleaned. Leakage sources may be looked for and repaired. Moreover other defects, such as lens element shifts, can be corrected. In case of fiber rupture in the fiber optics 12, this fiber optics may be replaced. The subsequent re-assembly is carried out in reverse order.

1. A rigid endoscope (1) comprising a fiber optics (12) running within a guide tube (5) that is rigidly connected to the endoscope (1), objective (14) that is mounted in the guide tube (6) in front of a distal end of the fiber optics (12) and an ocular (22) that is mounted in front of a proximal end of the fiber optics wherein
the fiber optics (12) rests in an axially displaceable manner in the guide tube (5) and is detachably affixed to the endoscope at the proximal end (15).

2. The endoscope as claimed in claim 1, wherein the distal end of the fiber optics (12) rests against a stop (6) at the guide tube (5) and is axially forced toward said stop at a proximal affixation (18) by a spring clip (19) toward the said stop.

3. The endoscope as claimed in claim 1, wherein the objective (14) rests in an objective tube (13) which, in turn, rests in an axially displaceable manner in the guide tube (5) and is affixed to the distal end of the fiber optics (12).

4. The endoscope as claimed in claim 1, wherein the proximal end of the fiber optics (12) is affixed to a support (15) that, in turn, is detachably mounted on the endoscope (8).

5. The endoscope as claimed in claim 2, wherein the support (15) rests in an axially displaceable manner in the endoscope (8) and is loaded by spring clips (19).

6. The endoscope as claimed in claim 4, wherein the support (15) bears a window (16) which covers the proximal end of the fiber optics (12).

7. The endoscope as claimed in claim 4, wherein the support (15) rests in an axially displaceable manner in the endoscope (8) and is loaded by spring clips (19).
